(19) United States
(12) Patent Application Publication
(10) Pub. No.: US 2023/0107609 A1
(43) Pub. Date: Apr. 6, 2023

(54) ANTI-PD-1 ANTIBODIES AND METHODS OF USE

(71) Applicant: CureImmune Therapeutics Inc., Vancouver (CA)

(72) Inventors: Yiwen LI, Woodcliff Lake, NJ (US); Yuxiang HU, Richmond (CA)

(21) Appl. No.: 17/913,752

(22) PCT Filed: Mar. 25, 2021
(86) PCT No.: PCT/US2021/024208

§ 371 Date: Sep. 22, 2022

Related U.S. Application Data
(60) Provisional application No. 63/000,386, filed on Mar. 26, 2020.

(51) Int. Cl. C07K 16/28; A61P 35/00

(52) U.S. Cl. CPC C07K 16/2896; A61P 35/00; C07K 2317/92; C07K 2317/565; C07K 2317/24; C07K 2317/52; C07K 2317/70

(57) ABSTRACT

The present disclosure provides anti-PD-1 antibodies or antigen binding fragments thereof, compositions comprising the antibodies or antigen binding fragments thereof, and methods of treating cancer or viral infection with the antibodies or antigen binding fragments thereof.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 220096_401WO_SEQUENCE_LISTING.txt. The text file is 35.0 KB, was created on Mar. 22, 2021, and is being submitted electronically via EFS-Web

### BACKGROUND

Cancer immunotherapy aims to induce an effective immune response that will discriminate between malignant tumor cells and normal cells. Cancer is caused by the uncontrolled growth of transformed cells. Cancer cells may express tumor neoantigens which can be recognized by immune cells as non-self. T cells play a central role in mediating anti-tumor immune response. Full activation of T cells requires two signals. The first signal is received by T cell receptor (TCR) on T cells upon binding of a small peptide antigen presented in the context of major histocompatibility complex (MHC) by professional antigen presenting cells (APC), such as macrophages and dendritic cells (DC). The second signal is delivered by costimulatory molecules (such as CD80) on APC to their corresponding receptors (e.g., CD28) on T cells. Once T cells become fully activated, a set of co-inhibitory molecules (such as CTLA-4 and PD-1) are upregulated on T cells to tune down the activation level in order to avoid unwanted damage to normal tissues.

PD-1 (programmed cell death protein-1, also known as CD279) is a type I protein of the CD28/CTLA-4 family of T cell regulators. It is composed of an extracellular IgV domain, a transmembrane domain and an intracellular domain containing an immunoreceptor tyrosine-based inhibitory motif (ITIM) and an immunoreceptor tyrosine-based switch motif (ITSM). PD-1 is mainly expressed on T cells, and can also be expressed on NK cells and myeloid cells. There are two natural ligands for PD-1. PD-L1 and PD-L2 bind to PD-1 to induce signaling through ITIM and ITSM to negatively regulate TCR signals. PD-L1 is expressed on macrophages and mature dendritic cells (DC). PD-L2 expression is more restricted and is expressed mainly by DCs.

The tumor has adopted many different mechanisms to escape immune recognition and destruction. PD-L1and/or PD-L2 can be expressed by tumor cells or tumor-infiltrating myeloid cells to interact with PD-1, dampening antitumor T cell responses.

Blocking the PD-1/PD-L1 axis has emerged as a new paradigm for the treatment of cancer. However, only a subset of cancer patients responds to PD-1/PD-L1 blockade therapy. Most of patients remain refractory, or acquire resistance to such therapy.

### BRIEF SUMMARY

In one aspect, the present disclosure provides an isolated antibody, or an antigen-binding fragment thereof, that specifically binds to PD-1, wherein the antibody or antigen-binding fragment thereof comprises a heavy chain variable region (VH) and a light chain variable region (VL), wherein the VH comprises a heavy chain CDR1 (VH-CDR1) comprising the amino acid sequence of SEQ ID NO:1, a VH-CDR2 comprising the amino acid sequence of SEQ ID NO:2, a VH-CDR3 comprising the amino acid sequence of SEQ ID NO:3; and the VL comprises a CDR1 (VL-CDR1) comprising the amino acid sequence of SEQ ID NO:4, a VL-CDR2 comprising the amino acid sequence of SEQ ID NO:5, and a VL-CDR3 comprising the amino acid sequence of SEQ ID NO:6; or the VH comprises a heavy chain CDR1 (VH-CDR1) comprising the amino acid sequence of SEQ ID NO:7, a VH-CDR2 comprising the amino acid sequence of SEQ ID NO:8, a VH-CDR3 comprising the amino acid sequence of SEQ ID NO:9; and the VL comprises a CDR1 (VL-CDR1) comprising the amino acid sequence of SEQ ID NO:10, a VL-CDR2 comprising the amino acid sequence of SEQ ID NO:11, and a VL-CDR3 comprising the amino acid sequence of SEQ ID NO:12.

In another aspect, the present disclosure provides a pharmaceutical composition comprising an antibody or antigen binding fragment thereof that specifically binds to PD-1 disclosed herein and a pharmaceutically acceptable carrier.

In another aspect, the present disclosure provides an isolated nucleic acid that encodes a heavy chain or light chain of an anti-PD-1 antibody or antigen binding fragment thereof. The present disclosure also provides a vector comprising the isolated nucleic acid, an isolated host cell comprising the isolated nucleic acid or the vector, an isolated host cell that expresses an anti-PD-1 antibody or antigen binding fragment disclosed herein, and a method of producing an anti-PD-1 antibody comprising culturing the host cell disclosed herein under conditions suitable for expressing the antibody or antigen binding fragment thereof.

In a further aspect, the present disclosure provides a method of treating cancer or a viral infection comprising administering to a patient an effective amount of an anti-PD-1 antibody or antigen binding fragment thereof, or a pharmaceutical composition of comprising the antibody or antigen binding fragment thereof disclosed herein.

### BRIEF DESCRIPTION OF THE SEVERAL VIEWS OF THE DRAWINGS

FIGS. 1A-1B. Binding activities of PD-1 antibodies in ELISA. 96-well plates were coated with 25 ng/well of recombinant (FIG. 1A) human, (FIG. 1B) cynomolgus PD-1-Fc fusion protein. Bound antibodies were detected using HRP-conjugated anti-human IgG Fab antibody and chromogenic substrate (OD at 450 nm).

FIG. 2. Flow cytometry analysis of PD-1 transfected Jurkat cells by PD-1 antibodies. PD-1 transfected Jurkat cells were stained with anti-PD-1 antibodies or isotype control IgG1 followed by BB700-labeled anti-Fab antibody.

FIGS. 3A-3B. Blocking activities of PD-1 antibodies in ELISA. Serially diluted PD-1 antibodies or control IgG were mixed with a fixed amount of biotinylated PD-1-Fc fusion protein. The mixture was transferred to 96-well plates pre-coated with recombinant human PD-L1-Fc (FIG. 3A) or with human PD-L2-Fc fusion protein (FIG. 3B) and then incubated for 1 h. Plates were washed, Streptavidin-HRP conjugate was then added. The absorbance at 450 nm was measured.

FIG. 4. Enhancement of T cell activation by PD-1 antibodies in a mixed leukocyte reaction assay. CD4⁺ T cells were co-cultured with allogeneic dendritic cells in the presence of a titration of PD-1 antibodies or isotype control antibody for 5 days. Supernatants were measured for IFN-γ production by ELISA.

FIG. 5. Enhancement of T cell activation by PD-1 antibodies in a PD1 luciferase reporter assay. PD-L1 aAPC/CHO-K1 human T-activator cells were co-cultured with GloResponse NFAT-1uc2/PD1 Jurkat cells in the presence or absence of PD-1 antibodies or control IgG for 6 hours. Luminescence activity was measured in a plate reader.

FIG. 6. PD-1 antibody inhibits tumor growth in a syngeneic MC38 tumor model in human PD-1 knock-in C57BL/6 mice. Mice were inoculated subcutaneously with MC38 tumor cells. When tumors reached an average volume of ˜100 mm³, groups of mice were treated with intraperitoneal injection of PD-1 antibodies or control IgG at 10 mg/kg, twice weekly (as indicated by an arrow). Tumor growth was monitored by measurement.

### DETAILED DESCRIPTION

The present disclosure provides anti-PD-1 antibodies or antigen-binding fragments thereof, which are useful in treating cancer, optionally in combination with other anti-cancer therapeutic agents, or viral infections, optionally in combination with anti-viral agents.

Anti-PD-1 antibodies or antigen-binding fragments thereof as provided herein are capable of binding to PD-1 expressing cells. The anti-PD-1 antibodies are also capable of blocking receptor-ligand interaction of PD-1 to PD-L1 and enhancing T cell activation in vitro and anti-tumor activity of an anti-cancer therapeutic in vivo. Surprisingly, certain anti-PD-1 antibodies, while blocking PD-1 interaction with PD-L1, do not significantly block PD-1 interaction with PD-L2.

Prior to setting forth this disclosure in more detail, it may be helpful to an understanding thereof to provide definitions of certain terms used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer) or subranges, unless otherwise indicated.

As used herein, the term “about” means ±20% of the indicated range, value, or structure, unless otherwise indicated.

It should be understood that the terms “a” and “an” as used herein refer to “one or more” of the enumerated components. The use of the alternative (e.g., “or”) should be understood to mean either one, both, or any combination thereof of the alternatives.

As used herein, the terms “include,” “have,” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not.

As used herein, “PD-1” or “programmed cell death protein 1” or “CD279” refers to an immune checkpoint molecule expressed on the surface of T cells, B cells, and macrophages that has a role in down-regulating the immune system by promoting apoptosis of antigen-specific T cells in lymph nodes and reducing apoptosis of regulatory T cells. PD-1 is a type I membrane protein and member of the CD28/CTLA-4 family of T cell regulators. The PD-1 protein includes an extracellular IgV domain, followed by a transmembrane region, and intracellular region. The intracellular region contains two phosphorylation sites located in an immunoreceptor tyrosine-based inhibitory motif and an immunoreceptor tyrosine-based switch motif. PD-1 binds to two ligands, PD-L1 and PD-L2. PD-1 includes mammalian PD-1 proteins, e.g., human and non-human primate. In some embodiments, PD-1 is a human PD-1 (NCBI Reference Sequence NP_005009) or cynomologus monkey PD-1 (Genbank Accession No. ABR15751).

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

As used herein, “protein” or “polypeptide” as used herein refers to a compound made up of amino acid residues that are covalently linked by peptide bonds. The term “protein” may be synonymous with the term “polypeptide” or may refer, in addition, to a complex of two or more polypeptides. A polypeptide may further contain other components (e.g., covalently bound), such as a tag, a label, a bioactive molecule, or any combination thereof. In certain embodiments, a polypeptide may be a fragment. As used herein, a “fragment” means a polypeptide that is lacking one or more amino acids that are found in a reference sequence. A fragment can comprise a binding domain, antigen, or epitope found in a reference sequence. A fragment of a reference polypeptide can have at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more of amino acids of the amino acid sequence of the reference sequence.

As described herein, a “variant” polypeptide species has one or more non-natural amino acids, one or more amino acid substitutions, one or more amino acid insertions, one or more amino acid deletions, or any combination thereof at one or more sites relative to a reference polypeptide as presented herein. In certain embodiments, “variant” means a polypeptide having a substantially similar activity (e.g., enzymatic function, immunogenicity) or structure relative to a reference polypeptide). A variant of a reference polypeptide can have at least about 40%, 45%, 50%, 55%, 60%, 65%, 70%, 75%, 80%, 85%, 90%, 91%, 92%, 93%, 94%, 95%, 96%, 97%, 98%, 99%, or more sequence identity to the amino acid sequence for the reference polypeptide as determined by sequence alignment programs and parameters known in the art. The variant can result from, for example, a genetic polymorphism or human manipulation. Conservative substitutions of amino acids are well known and may occur naturally or may be introduced when a protein is recombinantly produced. Amino acid substitutions, deletions, and additions may be introduced into a protein using mutagenesis methods known in the art (see, e.g., Sambrook et al., Molecular Cloning: A Laboratory Manual, 3d ed., Cold Spring Harbor Laboratory Press, NY, 2001). Oligonucleotide-directed site-specific (or segment specific) mutagenesis procedures may be employed to provide an altered polynucleotide that has particular codons altered according to the substitution, deletion, or insertion desired. Alternatively, random or saturation mutagenesis techniques, such as alanine scanning mutagenesis, error prone polymerase chain reaction mutagenesis, and oligonucleotide-directed mutagenesis may be used to prepare polypeptide variants (see, e.g., Sambrook et al., supra).

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

The terms “identical” or “percent identity,” in the context of two or more polypeptide or nucleic acid molecule sequences, means two or more sequences or subsequences that are the same or have a specified percentage of amino acid residues or nucleotides that are the same over a specified region (e.g., 60%, 65%, 70%, 75%, 80%, 85%, 90%, 91%, 92%, 93%, 94%, 95%, 96%, 97%, 98%, 99%, or 100% identity), when compared and aligned for maximum correspondence over a comparison window, or designated region, as measured using methods known in the art, such as a sequence comparison algorithm, by manual alignment, or by visual inspection. The algorithm used herein for determining percent sequence identity and sequence similarity is the BLAST 2.0 algorithm, as described in Altschul et al. “Gapped BLAST and PSI-BLAST: a new generation of protein database search programs,” Nucleic Acids Res. 2007, 25, 3389-3402, with the parameters set to default values.

As used herein, a “fusion protein” comprises a single chain polypeptide having at least two distinct domains, wherein the domains are not naturally found together in a protein. A nucleic acid molecule encoding a fusion protein may be constructed using PCR, recombinantly engineered, or the like, or such fusion proteins can be made synthetically. A fusion protein may further contain other components (e.g., covalently bound), such as a tag, linker, or bioactive molecule.

A “nucleic acid molecule” or “polynucleotide” refers to a polymeric compound containing nucleotides that are covalently linked by 3′-5′ phosphodiester bonds. Nucleic acid molecules include polyribonucleic acid (RNA), polydeoxyribonucleic acid (DNA), which includes genomic DNA, mitochondrial DNA, cDNA, or vector DNA. A nucleic acid molecule may be double stranded or single stranded, and if single stranded, may be the coding strand or non-coding (anti-sense strand). A nucleic acid molecule may contain natural subunits or non-natural subunits. A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

Variants of the polynucleotides of this disclosure are also contemplated. Variant polynucleotides are at least 80%, 85%, 90%, 95%, 99%, or 99.9% identical to a reference polynucleotide as described herein, or that hybridizes to a reference polynucleotide of defined sequence under stringent hybridization conditions of 0.015M sodium chloride, 0.0015M sodium citrate at about 65° -68° C. or 0.015M sodium chloride, 0.0015M sodium citrate, and 50% formamide at about 42° C. The polynucleotide variants retain the capacity to encode an immunoglobulin-like binding protein or antigen-binding fragment thereof having the functionality described herein.

The term “isolated” means that the material is removed from its original environment (e.g., the natural environment if it is naturally occurring). For example, a naturally occurring polynucleotide or polypeptide present in a living animal is not isolated, but the same polynucleotide or polypeptide, separated from some or all of the co-existing materials in the natural system, is isolated. Such polynucleotide could be part of a vector and/or such polynucleotide or polypeptide could be part of a composition (e.g., a cell lysate), and still be isolated in that such vector or composition is not part of the natural environment for the nucleic acid or polypeptide.

As used herein, the term “engineered,” “recombinant,” or “non-natural” refers to an organism, microorganism, cell, nucleic acid molecule, or vector that includes at least one genetic alteration or has been modified by introduction of an exogenous or heterologous nucleic acid molecule, wherein such alterations or modifications are introduced by genetic engineering (i.e., human intervention). Genetic alterations include, for example, modifications introducing expressible nucleic acid molecules encoding functional RNA, proteins, fusion proteins or enzymes, or other nucleic acid molecule additions, deletions, substitutions, or other functional disruption of a cell's genetic material. Additional modifications include, for example, non-coding regulatory regions in which the modifications alter expression of a polynucleotide, gene, or operon.

As used herein, “heterologous” or “exogenous” nucleic acid molecule, construct or sequence refers to a nucleic acid molecule or portion of a nucleic acid molecule that is not native to a host cell, but may be homologous to a nucleic acid molecule or portion of a nucleic acid molecule from the host cell. The source of the heterologous or exogenous nucleic acid molecule, construct or sequence may be from a different genus or species. In certain embodiments, a heterologous or exogenous nucleic acid molecule is added (i.e., not endogenous or native) to a host cell or host genome by, for example, conjugation, transformation, transfection, electroporation, or the like, wherein the added molecule may integrate into the host genome or exist as extra-chromosomal genetic material (e.g., as a plasmid or other form of self-replicating vector), and may be present in multiple copies. In addition, “heterologous” refers to a non-native enzyme, protein, or other activity encoded by an exogenous nucleic acid molecule introduced into the host cell, even if the host cell encodes a homologous protein or activity.

As used herein, the term “endogenous” or “native” refers to a gene, protein, or activity that is normally present in a host cell. Moreover, a gene, protein or activity that is mutated, overexpressed, shuffled, duplicated or otherwise altered as compared to a parent gene, protein or activity is still considered to be endogenous or native to that particular host cell. For example, an endogenous control sequence from a first gene (e.g., promoter, translational attenuation sequences) may be used to alter or regulate expression of a second native gene or nucleic acid molecule, wherein the expression or regulation of the second native gene or nucleic acid molecule differs from normal expression or regulation in a parent cell.

As used herein, the term “expression”, refers to the process by which a polypeptide is produced based on the encoding sequence of a nucleic acid molecule, such as a gene. The process may include transcription, post-transcriptional control, post-transcriptional modification, translation, post-translational control, posttranslational modification, or any combination thereof. An expressed nucleic acid molecule is typically operably linked to an expression control sequence (e.g., a promoter).

As described herein, more than one heterologous nucleic acid molecule can be introduced into a host cell as separate nucleic acid molecules, as a plurality of individually controlled genes, as a polycistronic nucleic acid molecule (e.g., a heavy chain and a light chain of an antibody), as a single nucleic acid molecule encoding a protein (e.g., a heavy chain of an antibody), or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

As used herein, the term “introduced” in the context of inserting a nucleic acid sequence into a cell, means “transfection”, or “transformation” or “transduction” and includes reference to the incorporation of a nucleic acid sequence into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

Additional definitions are provided in the sections below.

### Anti-PD-1 Antibodies or Antigen-Binding Fragments Thereof

In one aspect, antibodies (e.g., isolated monoclonal antibodies) or antigen-binding fragments thereof that specifically bind to PD-1, also referred to as anti-PD-1 antibodies or antigen-binding fragments thereof, are provided.

In some embodiments, antibodies or antigen-binding fragments thereof of the present disclosure specifically bind PD-1 with high affinity. As used herein, “specifically binds” or “specific for” may in some embodiments refer to an association or union of a binding protein (e.g., an anti-PD-1 antibody) or a binding domain (or fusion protein thereof) to a target molecule with an affinity or Kₐ (i.e., an equilibrium association constant of a particular binding interaction with units of 1/M) equal to or greater than 10⁵ M⁻¹ (which equals the ratio of the on-rate [kₒₙ] to the off-rate [k_{off}] for this association reaction), while not significantly associating or uniting with any other molecules or components in a sample. Binding domains (or fusion proteins thereof) may be classified as “high affinity” binding domains (or fusion proteins thereof) and “low affinity” binding domains (or fusion proteins thereof). “High affinity” binding domains refer to those binding domains with a Kₐ of at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹, preferably at least 10⁸ M⁻¹ or at least 10⁹ M⁻¹. “Low affinity” binding domains refer to those binding domains with a Kₐ of up to 10⁸ M⁻¹, up to 10⁷ M⁻¹, up to 10⁶ M⁻¹, up to 10⁵ M⁻¹. Alternatively, affinity may be defined as an equilibrium dissociation constant (K_{D}) of a particular binding interaction with units of M (e.g., 10⁻⁵ M to 10⁻¹³ M), (which equals the ratio of the off-rate [k_{off}] to the on-rate [kₒₙ] for this association reaction).

In some embodiments, antibodies or antigen-binding fragments thereof of the present disclosure bind to human PD-1 with a K_{D} of about 0.045 nM, with K_{off} of about 1.09×10⁻⁵ l/s and Kₒₙ of about 2.44×10⁻⁵ l/(M·s).

In some embodiments, antibodies or antigen-binding fragments thereof of the present disclosure bind to human PD-1 with a K_{D} of about 0.609 nM, with K_{off} of about 1.72×10⁻⁴ l/s and Kₒₙ of about 2.83×10⁻⁵ l/(M·s).

A variety of assays are known for identifying binding domains of the present disclosure that specifically bind a particular target, as well as determining binding domain or fusion protein affinities, such as Western blot, ELISA, analytical ultracentrifugation, spectroscopy and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent).

Terms understood by those in the art of antibody technology are each given the meaning acquired in the art, unless expressly defined differently herein. The term “antibody” refers to an intact antibody comprising at least two heavy (H) chains and two light (L) chains inter-connected by disulfide bonds, as well as any antigen-binding portion or fragment of an intact antibody that has or retains the ability to bind to the antigen target molecule recognized by the intact antibody, such as an scFv, Fab, or Fab′2 fragment. Thus, the term “antibody” herein is used in the broadest sense and includes polyclonal and monoclonal antibodies, including intact antibodies and functional (antigen-binding) antibody fragments thereof, including fragment antigen binding (Fab) fragments, F(ab′)2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody). The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific antibodies, diabodies, triabodies, tetrabodies, tandem di-scFv, and tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof (IgG1, IgG2, IgG3, IgG4), IgM, IgE, IgA, and IgD.

A monoclonal antibody or antigen-binding portion thereof may be non-human, chimeric, humanized, or human. Immunoglobulin structure and function are reviewed, for example, in Harlow et al., Eds., Antibodies: A Laboratory Manual, Chapter 14 (Cold Spring Harbor Laboratory, Cold Spring Harbor, 1988).

The terms “VL” and “VH” refer to the variable binding region from an antibody light chain and an antibody heavy chain, respectively. The variable binding regions comprise discrete, well-defined sub-regions known as “complementarity determining regions” (CDRs) and “framework regions” (FRs). The terms “complementarity determining region,” and “CDR,” are synonymous with “hypervariable region” or “HVR,” and refer to sequences of amino acids within antibody variable regions, which, in general, together confer the antigen specificity and/or binding affinity of the antibody, wherein consecutive CDRs (i.e., CDR1 and CDR2, CDR2 and CDR3) are separated from one another in primary amino acid sequence by a framework region. There are three CDRs in each variable region (HCDR1, HCDR2, HCDR3; LCDR1, LCDR2, LCDR3; also referred to as CDRHs and CDRLs, respectively). In certain embodiments, an antibody VH comprises four FRs and three CDRs as follows: FR1-HCDR1-FR2-HCDR2-FR3-HCDR3-FR4; and an antibody VL comprises four FRs and three CDRs as follows: FR1-LCDR1-FR2-LCDR2-FR3-LCDR3-FR4. In general, the VH and the VL together form the antigen-binding site through their respective CDRs.

Numbering of CDR and framework regions may be determined according to any known method or scheme, such as the Kabat, Chothia, EU, IMGT, and AHo numbering schemes (see, e.g., Kabat et al., “Sequences of Proteins of Immunological Interest, US Dept. Health and Human Services, Public Health Service National Institutes of Health, 1991, 5^{th} ed.; Chothia and Lesk, J. Mol. Biol. 196:901-917 (1987)); Lefranc et al., Dev. Comp. Immunol. 27:55, 2003; Honegger and Plückthun, J. Mol. Mo. 309:657-670 (2001)). Equivalent residue positions can be annotated and for different molecules to be compared using Antigen receptor Numbering And Receptor Classification (ANARCI) software tool (2016, Bioinformatics 15:298-300). Accordingly, identification of CDRs of an exemplary variable domain (VH or VL) sequence as provided herein according to one numbering scheme is not exclusive of an antibody comprising CDRs of the same variable domain as determined using a different numbering scheme. CDRs of the anti-PD-1 antibodies provided in the present disclosure are identified according to the IMGT numbering scheme unless indicated otherwise.

In some embodiments, an isolated antibody or an antigen-binding fragment thereof that specifically binds to PD-1 is provided, wherein the antibody or antigen-binding fragment thereof comprises a heavy chain variable region (VH) and a light chain variable region (VL), wherein the VH comprises a heavy chain CDR1 (VH-CDR1) comprising the amino acid sequence of SEQ ID NO:1, a heavy chain CDR2 (VH-CDR2) comprising the amino acid sequence of SEQ ID NO:2, and a heavy chain CDR3 (VH-CDR3) comprising the amino acid sequence of SEQ ID NO:3; and the VL comprises a light chain CDR1 (VL-CDR1) comprising the amino acid sequence of SEQ ID NO:4, a light chain CDR2 (VL-CDR2) comprising the amino acid sequence of SEQ ID NO:5, and a light chain CDR3 (VL-CDR3) comprising the amino acid sequence of SEQ ID NO:6; or the VH comprises a VH-CDR1 comprising the amino acid sequence of SEQ ID NO:7, a VH-CDR2 comprising the amino acid sequence of SEQ ID NO:8, and a VH-CDR3 comprising the amino acid sequence of SEQ ID NO:9, and the VL comprises a VL-CDR1 comprising the amino acid sequence of SEQ ID NO:10, a VL-CDR2 comprising the amino acid sequence of SEQ ID NO:11, and a VL-CDR3 comprising the amino acid sequence of SEQ ID NO:12. In some such embodiments, the VH comprises an amino acid sequence that has at least 90%, 91%, 92%, 93%, 94%, 95%, 96%, 97%, 98%, or 99% identity with the amino acid sequence of SEQ ID NO:13, and a VL comprises an amino acid sequence that has at least 90%, 91%, 92%, 93%, 94%, 95%, 96%, 97%, 98%, or 99% identity with the amino acid sequence of SEQ ID NO:15, provided that the amino acid sequences of the VH-CDRs (SEQ ID NOS:1-3) and VL-CDRs (SEQ ID NOS:4-6) are unchanged; or the VH comprises an amino acid sequence that has at least 90% identity with the amino acid sequence of SEQ ID NO:17, and a VL comprises an amino acid sequence that has at least 90%, 91%, 92%, 93%, 94%, 95%, 96%, 97%, 98%, or 99% identity with the amino acid sequence of SEQ ID NO:19, provided that the amino acid sequences of the VH-CDRs (SEQ ID NOS:7-9) and VL-CDRs (SEQ ID NOS:10-12) are unchanged.

In some embodiments, the anti-PD-1 antibody or antigen binding fragment thereof comprises: (a) a VH comprising the amino acid sequence of SEQ ID NO:13, and a VL comprising the amino acid sequence of SEQ ID NO:15; or (b) a VH comprising the amino acid sequence of SEQ ID NO:17, and a VL comprising the amino acid sequence of SEQ ID NO:19.

In some embodiments, an anti-PD-1 antibody of the present disclosure comprises a heavy chain (HC) and a light chain (LC). The heavy chain typically comprises a VH and a heavy chain constant region (CH). Depending on the antibody isotype from which it derives, a heavy chain constant region may comprise CH1, CH2, and CH3 domains (IgG). In some embodiments, the heavy chain constant region comprises a human IgG1, IgG2, IgG3, or IgG4 constant region. An exemplary human IgG1 heavy chain constant region amino acid sequence comprises an amino acid sequence of SEQ ID NO:29. Another exemplary human IgG1 heavy chain constant region amino acid sequence comprises an amino acid sequence of SEQ ID NO:21. The light chain typically comprises a VL and a light chain constant region (CL). In some embodiments, a CL comprises a C kappa (“CK”) constant region. In some embodiments, a CL comprises a C lambda (Cλ) constant region. An exemplary human light chain C kappa constant region nucleic acid sequence comprises a nucleic acid sequence of SEQ ID NO:24. An exemplary human light chain C kappa constant region amino acid sequence comprises an amino acid sequence of SEQ ID NO:23. In some embodiments, an anti-PD-1 antibody of the present disclosure comprises two heavy chains and two light chains, held together covalently by disulfide bridges.

In some embodiments, the antibody or antigen-binding fragment of the present disclosure comprises a CL comprising an amino acid sequence having 90%, 91%, 92%, 93%, 94%, 95%, 96%, 97%, 98%, 99%, or 100% identity to the amino acid sequence of SEQ ID NO:23. In some embodiments, an antibody or antigen-binding fragment of the present disclosure comprises an IgG1 heavy chain constant region comprising an amino acid sequence having 90%, 91%, 92%, 93%, 94%, 95%, 96%, 97%, 98%, 99%, or 100% identity to the amino acid sequence of SEQ ID NO:29. In some embodiments, an antibody or antigen-binding fragment of the present disclosure comprises an IgG1 heavy chain constant region comprising an amino acid sequence having 90%, 91%, 92%, 93%, 94%, 95%, 96%, 975, 98%, 99%, or 100% identity to the amino acid sequence of SEQ ID NO:21. In some embodiments, an antibody or antigen-binding fragment of the present disclosure comprises an IgG4 constant region comprising an amino acid sequence having 90%, 91%, 92%, 93%, 94%, 95%, 96%, 97%, 98%, 99%, or 100% identity to the amino acid sequence of SEQ ID NO:33.

In some embodiments, the antibody or antigen-binding fragment of the present disclosure comprises a Fc region portion. As used herein, “Fc region portion” refers to the heavy chain constant region segment of the Fc fragment (the “fragment crystallizable” region or Fc region) from an antibody, which can include one or more constant domains, such as CH2, CH3, or both. In some embodiments, an Fc region portion includes the CH2 and CH3 domains of an IgG antibody. In some embodiments, a CH2CH3 structure has sub-region domains from the same antibody isotype and are human, such as human IgG1, IgG2, IgG3, or IgG4 (e.g., CH2CH3 from human IgG1). By way of background, an Fc region is responsible for the effector functions of an antibody, such as ADCC (antibody-dependent cell-mediated cytotoxicity), CDC (complement-dependent cytotoxicity) and complement fixation, binding to Fc receptors (e.g., CD16, CD32, FcRn), greater half-life in vivo relative to a polypeptide lacking an Fc region, protein A binding, and perhaps even placental transfer (see Capon et al. Nature 337: 525, 1989). In some embodiments, a Fc region portion in an antibody or antigen-binding fragment of the present disclosure will be capable of mediating one or more of these effector functions. In some embodiments, a Fc region portion in an antibody or antigen-binding fragment of the present disclosure has normal effector function, meaning having less than 25%, 20%, 15%, 10%, 5%, 1% difference in effector function (e.g., ADCC, CDC, or both) as compared to a wildtype IgG1 antibody.

In some embodiments, a Fc region portion in an antibody or antigen-binding fragment of the present disclosure has a reduction in one or more of these effector functions or lack one or more effector functions by way of, for example, one or more amino acid substitutions or deletions in the Fc region portion known in the art. An antibody or antigen-binding fragment having a mutated or variant Fc region portion having reduced effector function means that the antibody exhibits a decrease of at least 20%, 25%, 30%, 35%, 40%, 45%, 50%, 55%, 60%, 65%, 70%, 75%, 80%, 85%, 90%, 95%, or 99% in FcR binding, ADCC, CDC, or any combination thereof, as compared to an antibody having a wildtype Fc region portion. In some embodiments, the human wildtype IgG1 Fc region comprises the amino acid sequence of SEQ ID NO:35. In some embodiments, the mutated or variant Fc region portion exhibits decreased binding to FcγRI (CD64), FcγRIIA (CD32), FcγRIIIA (CD16a), FcγRIIIB (CD16b), or any combination thereof. In some embodiments, the Fc region portion in an antibody or antigen-binding fragment of the present disclosure is a variant Fc region portion having reduced ADCC, CDC, or both. In some embodiments, the Fc region portion is a variant IgG1 Fc region portion comprising a mutation corresponding to amino acid E233P, L234V, L234A, L235A, L235E, AG236, G237A, E318A, K320A, K322A, A327G, P329G, A330S, P331S, or any combination thereof, as numbered according to the EU set forth in Kabat. For example, amino acid substitutions L234A, L235E, G237A introduced into an IgG1 Fc region portion reduces binding to FcγRI, FcγRIIa, and FcγRIII receptors, and A330S and P331S introduced into an IgG1 Fc region portion reduces C1q-mediated complement fixation. In some embodiments, the Fc region portion is a variant IgG1 Fc region portion comprising mutations corresponding to E233P, L234V, L235A, ΔG236, A327G, A330S, and P331S, as numbered according to the EU set forth in Kabat. In some embodiments, a variant IgG1 Fc region portion comprising mutations corresponding to E233P, L234V, L235A, ΔG236, A327G, A330S, and P331S comprises the amino acid sequence of SEQ ID NO:36.

In some embodiments, the antibody or antigen-binding fragment thereof of the present disclosure is glycosylated. IgG subtype antibodies contain a conserved glycosylation site at amino acid N297 in the CH2 domain of the Fc region portion. In some such embodiments, the Fc region portion in an antibody or antigen-binding fragment of the present disclosure comprises a N297 as numbered according to EU set forth in Kabat. In some embodiments, the antibody or antigen-binding fragment of the present disclosure comprises a mutation that alters glycosylation at N297 in the Fc region portion, optionally wherein the mutation that alters glycosylation comprises N297A, N297Q, or N297G. In some embodiments, an antibody or antigen-binding fragment thereof comprising a N297A, N297Q, or N297G mutation exhibits reduced Fc interaction with one or more low affinity FcγR(s), reduced CDC, reduced ADCC, or any combination thereof.

In some embodiments, the antibody or antigen-binding fragment of the present disclosure comprises a heavy chain (HC) and a light chain (LC), wherein the HC comprises an amino acid sequence that has at least 90%, 91%, 92%, 93%, 94%, 95%, 96%, 97%, 98%, 99%, or 100% identity with the amino acid sequence of SEQ ID NO:25, and the LC comprises an amino acid sequence that has at least 90%, 91%, 92%, 93%, 94%, 95%, 96%, 97%, 98%, or 99% identity with the amino acid sequence of SEQ ID NO:26; or the HC comprises an amino acid sequence that has at least 90%, 91%, 92%, 93%, 94%, 95%, 96%, 97%, 98%, or 99% identity with the amino acid sequence of SEQ ID NO:27, and the LC comprises an amino acid sequence that has at least 90%, 91%, 92%, 93%, 94%, 95%, 96%, 97%, 98%, or 99% identity with the amino acid sequence of SEQ ID NO:28.

In some embodiments, the antibody or antigen-binding fragment of the present disclosure comprises a HC comprising the amino acid sequence of SEQ ID NO:25, and a LC comprising the amino acid sequence of SEQ ID NO:26; or a HC comprising the amino acid sequence of SEQ ID NO:27, and a LC comprising the amino acid sequence of SEQ ID NO:28.

In any of the presently disclosed embodiments, the antibody or antigen-binding fragment comprises a Fc polypeptide or a fragment thereof, including a CH2 (or a fragment thereof, a CH3 (or a fragment thereof), or a CH2 and a CH3, wherein the CH2, the CH3, or both can be of any isotype and may contain amino acid substitutions or other modifications as compared to a corresponding wild-type CH2 or CH3, respectively. In certain embodiments, a Fc polypeptide of the present disclosure comprises two CH2-CH3 polypeptides that associate to form a dimer.

As used herein, unless otherwise provided, a position of an amino acid residue in the constant region of human IgG1 heavy chain is numbered assuming that the variable region of human IgG1 is composed of 128 amino acid residues according to the Kabat numbering convention. The numbered constant region of human IgG1 heavy chain is then used as a reference for numbering amino acid residues in constant regions of other immunoglobulin heavy chains. A position of an amino acid residue of interest in a constant region of an immunoglobulin heavy chain other than human IgG1 heavy chain is the position of the amino acid residue in human IgG1 heavy chain with which the amino acid residue of interest aligns. Alignments between constant regions of human IgG1 heavy chain and other immunoglobulin heavy chains may be performed using software programs known in the art, such as the Megalign program (DNASTAR Inc.) using the Clustal W method with default parameters. According to the numbering system described herein, for example, although human IgG2 C_{H2} region may have an amino acid deletion near its amino-terminus compared with other C_{H2} regions, the position of the “N” located at 296 in human IgG2 C_{H2} is still considered position 297 because this residue aligns with “N” at position 297 in human IgG1 C_{H2}.

In addition, antibodies have a hinge sequence that is typically situated between the Fab and Fc region (but a lower section of the hinge may include an amino-terminal portion of the Fc region). By way of background, an immunoglobulin hinge acts as a flexible spacer to allow the Fab portion to move freely in space. In contrast to the constant regions, hinges are structurally diverse, varying in both sequence and length between immunoglobulin classes and even among subclasses. For example, a human IgG1 hinge region is freely flexible, which allows the Fab fragments to rotate about their axes of symmetry and move within a sphere centered at the first of two inter-heavy chain disulfide bridges. By comparison, a human IgG2 hinge is relatively short and contains a rigid poly-proline double helix stabilized by four inter-heavy chain disulfide bridges, which restricts the flexibility. A human IgG3 hinge differs from the other subclasses by its unique extended hinge region (about four times as long as the IgG1 hinge), containing 62 amino acids (including 21 prolines and 11 cysteines), forming an inflexible poly-proline double helix and providing greater flexibility because the Fab fragments are relatively far away from the Fc fragment. A human IgG4 hinge is shorter than IgG1 but has the same length as IgG2, and its flexibility is intermediate between that of IgG1 and IgG2. Immunoglobulin structure and function are reviewed, for example, in Harlow et al., Eds., Antibodies: A Laboratory Manual, Chapter 14 (Cold Spring Harbor Laboratory, Cold Spring Harbor, 1988). An example of a human IgG1 hinge sequence for use in the anti-PD-1 antibody or antigen binding fragment of the present disclosure comprises the amino acid sequence of SEQ ID NO:34.

In some embodiments, the anti-PD-1 antibody or antigen binding fragment thereof of the present disclosure is chimeric, humanized, or human.

In some embodiments, the anti-PD-1 antibody or antigen-binding fragment thereof of blocks interaction of PD-1 and PD-L1. In some embodiments, the anti-PD-1 antibody or antigen-binding fragment thereof of blocks interaction of PD-1 and PD-L1, and PD-1 and PD-L2. In some embodiments, anti-PD-1 antibodies or antigen-binding fragments thereof of the present disclosure block interaction of PD-1 and PD-L1 without significantly blocking interaction of PD-1 and PD-L2. The ability of an antibody or antigen-binding fragment thereof to block interaction of PD-1 with its ligands PD-L1 and PD-L2 can be determined by ELISA as described in Example 1.

In some embodiments, the anti-PD-1 antibody or antigen-binding fragment thereof enhances T-cell activation. T cell activation can be measured using a mixed lymphocyte reaction (MLR) assay and measuring IFNγ production, or a recombinant Jurkat T cell expressing luciferase under the control of NFAT response elements with constitutive expression of human PD-1 as described in Example 1.

### Nucleic Acids, Vectors, and Host Cells

In another aspect, the present disclosure provides an isolated nucleic acid that encodes the anti-PD-1 antibody or antigen binding fragment thereof as described herein. In some embodiments, the isolated nucleic acid encodes the VH, the VL, or both the VH and VL of the antibody or antigen binding fragment thereof. In some embodiments, the isolated nucleic acid encodes the heavy chain, the light chain, or both the heavy and light chain of the antibody or antigen binding fragment thereof. In some embodiments, the nucleic acid encoding the anti-PD-1 antibody or antigen binding fragment thereof is codon optimized to enhance or maximize expression in certain types of cells (e.g., Scholten et al., Clin. Immunol. 119: 135-145, 2006). As used herein a “codon optimized” polynucleotide is a heterologous polypeptide having codons modified with silent mutations corresponding to the abundances of host cell tRNA levels.

In some embodiments, a nucleic acid molecule encoding an anti-PD-1 antibody or antigen binding fragment thereof of the present disclosure (e.g., an antibody heavy chain and light chain, or VH and VL regions) comprises a nucleic acid sequence for a heavy chain or VH region and a light chain or VL, respectively, wherein the heavy chain or VH region is separated from the light chain or VL region by a 2A self-cleaving peptide. In some embodiments, the 2A self-cleaving peptide is a porcine teschovirus-1 (P2A), equine rhinitis A virus (E2A), Thosea asigna virus (T2A), foot-and-mouth disease virus (F2A), or any combination thereof (see, e.g., Kim et al., PLOS One 6:e18556, 2011, which 2A nucleic acid and amino acid sequences are incorporated herein by reference in their entirety).

In another aspect, an expression construct comprising a nucleic acid encoding an anti-PD-1 antibody or antigen binding fragment thereof as described herein is provided. In some embodiments, a nucleic acid may be operably linked to an expression control sequence (e.g., expression construct). As used herein, “expression construct” refers to a DNA construct containing a nucleic acid molecule that is operably-linked to a suitable control sequence capable of effecting the expression of the nucleic acid molecule in a suitable host. An expression construct may be present in a vector (e.g., a bacterial vector, a viral vector) or may be integrated into a genome. The term “operably linked” refers to the association of two or more nucleic acids on a single polynucleotide fragment so that the function of one is affected by the other. For example, a promoter is operably-linked with a coding sequence when it is capable of affecting the expression of that coding sequence (i.e., the coding sequence is under the transcriptional control of the promoter). The term “expression control sequence” (also called a regulatory sequence) refers to nucleic acid sequences that effect the expression and processing of coding sequences to which they are operably linked. For example, expression control sequences may include transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion.

In some embodiments, a nucleic acid or an expression construct encoding an anti-PD-1 antibody or antigen binding fragment thereof is present in a vector. A “vector” is a nucleic acid molecule that is capable of transporting another nucleic acid. Vectors may be, for example, plasmids, cosmids, viruses, a RNA vector or a linear or circular DNA or RNA molecule that may include chromosomal, non-chromosomal, semi-synthetic or synthetic nucleic acids. Exemplary vectors are those capable of autonomous replication (episomal vector) or expression of nucleic acids to which they are linked (expression vectors). Exemplary viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996). In some embodiments, a vector is a plasmid. In some other embodiments, a vector is a viral vector. In some such embodiments, the viral vector is a lentiviral vector or a γ-retroviral vector.

In a further aspect, the present disclosure also provides an isolated host cell comprising a nucleic acid, expression construct, or vector encoding an anti-PD-1 antibody or antigen binding fragment thereof as described herein. As used herein, the term “host” refers to a cell or microorganism targeted for genetic modification with a heterologous or exogenous nucleic acid molecule to produce a polypeptide of interest (e.g., an anti-PD-1 antibody or antigen-binding fragment thereof). In certain embodiments, a host cell may optionally already possess or be modified to include other genetic modifications that confer desired properties related or unrelated to biosynthesis of the heterologous or exogenous protein (e.g., inclusion of a selectable marker). More than one heterologous or exogenous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof. When two or more exogenous nucleic acid molecules are introduced into a host cell, it is understood that the two more exogenous nucleic acid molecules can be introduced as a single nucleic acid molecule (e.g., on a single vector), on separate vectors, integrated into the host chromosome at a single site or multiple sites. The number of referenced heterologous nucleic acid molecules or protein activities refers to the number of encoding nucleic acid molecules or the number of protein activities, not the number of separate nucleic acid molecules introduced into a host cell.

Examples of host cells include, but are not limited to, eukaryotic cells, e.g., yeast cells, animal cells, insect cells, plant cells; and prokaryotic cells, including E. coli. In some embodiments, the cells are mammalian cells. In some embodiments, the host cell is a human embryonic kidney (HEK293) cell, Y0 cell, Sp2/0 cell, NSO murine myeloma cell, PER.C6® human cell, baby hamster kidney cell (BHK), COS cell, or Chinese hamster ovary (CHO) cell. Host cells are cultured using methods known in the art.

In some embodiments, the present disclosure provides a mammalian host cell comprising: a polynucleotide sequence encoding a polypeptide having the amino acid sequence of SEQ ID NO:13 and a polynucleotide sequence encoding a polypeptide having the amino acid sequence of SEQ ID NO:15; a polynucleotide sequence encoding a polypeptide having the amino acid sequence of SEQ ID NO:17 and a polynucleotide sequence encoding a polypeptide having the amino acid sequence of SEQ ID NO:19; a polynucleotide sequence encoding a polypeptide having the amino acid sequence of SEQ ID NO:25 and a polynucleotide sequence encoding a polypeptide having the amino acid sequence of SEQ ID NO:26; or a polynucleotide sequence encoding a polypeptide having the amino acid sequence of SEQ ID NO:27 and a polynucleotide sequence encoding a polypeptide having the amino acid sequence of SEQ ID NO:28, wherein the cell is capable of expressing an antibody or antigen-binding fragment thereof that binds human PD-1. In some such embodiments, the mammalian cell comprises: a polynucleotide sequence comprising SEQ ID NO:14 and a polynucleotide sequence comprising SEQ ID NO:16; or a polynucleotide sequence comprising SEQ ID NO:18 and a polynucleotide sequence comprising SEQ ID NO:20.

In yet another aspect, the present disclosure provides a process for making an anti-PD-1 antibody or antigen binding fragment thereof as described herein, comprising culturing a host cell of the present disclosure, under suitable conditions and for a sufficient time to express the anti-PD-1 antibody or antigen binding fragment thereof, and optionally isolating the anti-PD-1 antibody or antigen binding fragment thereof from the culture. Purification of soluble antibodies or antigen binding fragments thereof may be performed according to methods known in the art.

### Pharmaceutical Compositions

In another aspect, the present disclosure provides a composition comprising an anti-PD-1 antibody or antigen binding fragment thereof as described herein and a pharmaceutically acceptable carrier, diluent, or excipient. Pharmaceutically acceptable carriers for diagnostic and therapeutic use are well known in the pharmaceutical art, and are described, for example, in Remington's Pharmaceutical Sciences, Mack Publishing Co. (A. R. Gennaro (Ed.), 18^{th} Edition, 1990) and in CRC Handbook of Food, Drug, and Cosmetic Excipients, CRC Press LLC (S. C. Smolinski, ed., 1992). Exemplary pharmaceutically acceptable carriers include any adjuvant, carrier, excipient, glidant, diluent, preservative, dye/colorant, surfactant, wetting agent, dispersing agent, suspending agent, stabilizer, isotonic agent, solvent, emulsifier, or any combination thereof. For example, sterile saline and phosphate buffered saline at physiological pH can be suitable pharmaceutically acceptable carriers. Preservatives, stabilizers, dyes or the like may also be provided in the pharmaceutical composition. In addition, antioxidants and suspending agents may also be used. Pharmaceutical compositions may also contain diluents such as water, buffers, antioxidants such as ascorbic acid, low molecular weight polypeptides (less than about 10 residues), proteins, amino acids, carbohydrates (e.g., glucose, sucrose, dextrins), chelating agents (e.g., EDTA), glutathione, and other stabilizers and excipients. Neutral buffered saline or saline mixed with nonspecific serum albumin are exemplary diluents.

The pharmaceutical compositions described herein can be formulated for oral, topical, transdermal, inhalation, parenteral, sublingual, buccal, rectal, vaginal, and intranasal administration. The term “parenteral”, as used herein, includes subcutaneous, intravenous, intramuscular, intrasternal, and intratumoral injection or infusion techniques.

In some embodiments, pharmaceutical compositions of the present invention are formulated in a single dose unit or in a form comprising a plurality of dosage units. Methods of preparing such dosage forms are known, or will be apparent, to those skilled in this art; for example, see Remington: The Science and Practice of Pharmacy, 20th Edition (Philadelphia College of Pharmacy and Science, 2000).

A pharmaceutical composition may be in the form of a solid, semi-solid or liquid. Solid compositions may include powders and tablets. In some embodiments, the pharmaceutical compositions described here are lyophilized or in powder form for re-constitution with a suitable vehicle, e.g., sterile water, before use. In some embodiments, the pharmaceutical compositions described herein is a suspension, solution, or emulsion.

### Therapeutic Uses

The anti-PD-1 antibodies or antigen-binding fragments thereof of the present disclosure may be used in a method of treating cancer, comprising administering to a patient in need thereof, an effective amount of an anti-PD-1 antibody or antigen binding fragment of the present disclosure, or a pharmaceutical composition comprising an anti-PD-1 antibody or antigen binding fragment of the present disclosure.

Patients or subjects that can be treated by anti-PD-1 antibodies or antigen-binding fragments thereof of the present disclosure include, but are not limited to, a mammal, such as human or non-human primates (e.g., monkeys and apes), a domesticated animal (e.g., laboratory animals, household pets, or livestock), non-domesticated animal (e.g., wildlife), dog, cat, rodent, mouse, hamster, cow, bird, chicken, fish, pig, horse, goat, sheep, rabbit, and any combination thereof. In some embodiments, the subject is human. The subject can be male or female and can be any suitable age, including infant, juvenile, adolescent, adult, and geriatric.

“Treat,” “treatment,” or “ameliorate” refers to medical management of a disease, disorder, or condition of a subject (e.g., a human or non-human mammal, such as a primate, horse, cat, dog, goat, mouse, or rat). In general, an appropriate dose or treatment regimen comprising an antibody or antigen binding fragment thereof, or composition of the present disclosure, is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; prolonged survival; or any combination thereof.

A “therapeutically effective amount” or “effective amount” of an antibody, antigen-binding fragment, or composition of this disclosure refers to an amount of the composition or molecule sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. When referring to an individual active ingredient, administered alone, a therapeutically effective amount refers to the effects of that ingredient or cell expressing that ingredient alone. When referring to a combination, a therapeutically effective amount refers to the combined amounts of active ingredients that result in a therapeutic effect, whether administered serially, sequentially, or simultaneously. A combination may comprise, for example, an anti-PD-1 antibody or antigen binding fragment thereof and an anti-tumor agent.

An appropriate dose, suitable duration, and frequency of administration of the compositions will be determined by such factors as the condition of the patient, size, weight, body surface area, age, sex, type and severity of the disease, particular therapy to be administered, particular form of the active ingredient, time and the method of administration, and other drugs being administered concurrently, which can readily be determined by a person skilled in the art.

Generally, a therapeutically effective daily dose of an antibody or antigen binding fragment is (for a 70 kg mammal) from about 0.001 mg/kg (i.e., 0.07 mg) to about 100 mg/kg (i.e., 7.0 g); preferably a therapeutically effective dose is (for a 70 kg mammal) from about 0.01 mg/kg (i.e., 0.7 mg) to about 50 mg/kg (i.e., 3.5 g); more preferably a therapeutically effective dose is (for a 70 kg mammal) from about 1 mg/kg (i.e., 70 mg) to about 25 mg/kg (i.e., 1.75 g).

An anti-PD-1 antibody or antigen binding fragment thereof may be administered one or more times over a given period of time. In some embodiments, a method comprises administering the anti-PD-1 antibody or antigen binding fragment thereof to the subject at least 2, 3, 4, 5, 6, 7, 8, 9, 10 times, or more.

In certain embodiments, a method comprises administering the anti-PD-1 antibody or antigen binding fragment thereof to the subject a plurality of times, wherein a second or successive administration is performed at about 28 days, 21 days, 14 days, 10 days, 7 days, 3 days, 1 day, or less following a first administration.

Anti-PD-1 antibodies or antigen-binding fragments thereof of the present disclosure may be administered to a subject by parenteral routes. In some embodiments, anti-PD-1 antibodies or antigen-binding fragments thereof are administered to a subject by subcutaneous, intravenous, intraarterial, subdural, intramuscular, intracranial, intrasternal, intratumoral, intraperitoneal, or infusion techniques.

Cancers that may be treated by the anti-PD-1 antibody or antigen binding fragment thereof provided in the present disclosure include hematologic malignancies and solid tumors. In some embodiments, a hematologic malignancy is a leukemia, lymphoma, or myeloma. In some embodiments, a leukemia is acute lymphoblastic leukemia, acute myelogenous leukemia, chronic lymphocytic leukemia, chronic myelogenous leukemia, acute monocytic leukemia, hairy cell leukemia, B-cell prolymphocytic leukemia, T-cell prolymphocytic leukemia, or juvenile myelomonocytic leukemia. In some embodiments, a lymphoma is Hodgkin's lymphoma; non-Hodgkin's lymphoma; Epstein-Barr virus-associated lymphoproliferative disease; Burkitt lymphoma; large B cell lymphoma, not otherwise specified; diffuse large B cell lymphoma associated with chronic inflammation; fibrin-associated diffuse large cell lymphoma; primary effusion lymphoma; plasmablastic lymphoma; extranodal NK/T cell lymphoma, nasal type; peripheral T cell lymphoma, not otherwise specified; angioimmunoblastic T cell lymphoma; follicular T cell lymphoma; or systemic T cell lymphoma of childhood. In some embodiments, a myeloma is multiple myeloma or myelodysplastic syndrome.

In some embodiments, the cancer is a Hodgkin's lymphoma, non-Hodgkin's lymphoma, multiple myeloma, leukemia, myelodysplastic syndrome, thymus cancer, malignant mesothelioma, pituitary tumor, thyroid tumor, melanoma, Merkel cell skin cancer, lung cancer, head and neck cancer, colorectal cancer, liver cancer, bile duct cancer, gallbladder cancer, pancreatic cancer, esophageal cancer, gastric cancer, small intestine cancer, anal cancer, kidney cancer, bladder cancer, prostate cancer, penile cancer, testicular cancer, breast cancer, ovarian cancer, cervical cancer, vaginal cancer, vulvar cancer, endometrial cancer, eye cancer, soft tissue sarcoma, hepatocellular carcinoma, brain tumor, or spinal cord tumor.

In some embodiments, an anti-PD-1 antibody or antigen binding fragment thereof described herein may be used in combination with one or more anti-tumor agents. In some embodiments, the one or more anti-tumor agents is administered simultaneously, separately, or sequentially. In some embodiments, an anti-tumor agent is a cellular immunotherapy, antibody therapy, immune checkpoint inhibitor therapy, hormone therapy, chemotherapeutic, targeted cancer therapy, cytokine therapy or any combination thereof. In some embodiments, a cellular immunotherapy comprises a TCR-T cell therapy, dendritic cell therapy, or chimeric antigen receptor (CAR)-T cell therapy, or any combination thereof. In some embodiments, an antibody therapy comprises an agonistic, immune enhancing antibody. In some embodiments, an antibody therapy comprises an antibody-drug conjugate. In some embodiments, an antibody therapy comprises bevacizumab, nimotuzumab, lapatinib, cetuximab, panitumumab, matuzumab, trastuzumab, nimotuzumab, zalutumumab, alemtuzumab, rituxmiab, magrolimab, or any combination thereof. In some embodiments, an immune checkpoint inhibitor therapy targets PD-L1, PD-L2, CD80, CD86, B7-H3, B7-H4, HVEM, adenosine, GALS, VISTA, CEACAM-1, CEACAM-3, CEACAM-5, PVRL2, PD-1, CTLA-4, BTLA, KIR, LAG3, TIM3, A2aR, CD244/2B4, CD160, TIGIT, LAIR-1, PVRIG/CD112R, CD47, SIRPα, or any combination thereof. In some embodiments, an immune checkpoint inhibitor therapy comprises ipilimumab, tremelimumab, pidilizumab, nivolumab, pembrolizumab, durvalumab, atezolizumab, avelumab, urelumab, lirilumab, or any combination thereof. In some embodiments, a hormone therapy comprises abiraterone, anastrozole, exemestane, fulvestrant, letrozole, leuprolide, tamoxifen, or any combination thereof. In some embodiments, a cytokine therapy comprises IFNα, IL-2, IFNγ, GM-CSF, IL-7, IL-12, IL-21, IL-15, or any combination thereof.

In some embodiments, a chemotherapeutic comprises an alkylating agent, a platinum based agent, a cytotoxic agent, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), a DNA repair inhibitor, or an apoptosis inducing agent. Examples of chemotherapeutic agents considered for use in combination therapies include vemurafenib, dabrafenib, trametinib, cobimetinib, anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Exemplary alkylating agents include nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Exemplary platinum based agents include carboplatin, cisplatin, oxaliplatin, nedaplatin, picoplatin, satraplatin, phenanthriplatin, and triplatin tetranitrate.

Exemplary apoptosis inducing agents include AMG-224, AMG-176, and AMG-232, and venetoclax.

Exemplary targeted cancer therapies, therapies that target specific molecules that are involved in tumor growth, progression, and metastasis (e.g., oncogenes), include angiogenesis inhibitors (e.g., a VEGF pathway inhibitors), tyrosine kinase inhibitors (e.g., an EGF pathway inhibitors), receptor tyrosine kinase inhibitors, growth factor inhibitors, GTPase inhibitors, serine/threonine kinase inhibitors, transcription factor inhibitors, B-Raf inhibitors, RAF inhibitors, MEK inhibitors, mTOR inhibitors, EGFR inhibitors, ALK inhibitors, ROS1 inhibitors, BCL-2 inhibitors, PI3K inhibitors, VEGFR inhibitors, BCR-ABL inhibitors, MET inhibitors, MYC inhibitors, ABL inhibitors, HER2 inhibitors, BTK inhibitors, H-RAS inhibitors, K-RAS inhibitors, PDGFR inhibitors, TRK inhibitors, c-KIT inhibitors, c-MET inhibitors, CDK4/6 inhibitors, FAK inhibitors, FGFR inhibitors, FLT3 inhibitors, IDH1 inhibitors, IDH2 inhibitors, PARP inhibitors, PDGFRA inhibitors, and RET inhibitors. In some embodiments, a targeted cancer therapy comprises bevacizumab, figitumumab, ramucirumab, ranibizumab, vemurafenib, dabrafenib, encorafenib, vorinostat, binimetinib, cobimetinib, refametinib, selumetinib, trametinib, ibrutinib, tirabrutinib, acalabrutinib, spebrutinib, entrectinib, larotrectinib, lestaurtinib, imatinb, sunitinb, ponatinib, capmatinib, crizotinib, tivantinib, onartuzumab, savolitinib, tepotinib, palbociclib, ribociclib, abemaciclib, trilaciclib, defactinib, erdafitinib, pemigatinib, infigratinib, rogaratinib, quizartinib, crenolanib, gilteritinib, midostaurin, lestaurtinib, ivosidenib, enasidenib, talazoparib, niraparib, rucaparib, olaparib, veliparib, regorafenib, crenolanib, olaratumab, belvarafenib, lenvatinib, alectinib, vandetanib, cabozantinib, ceritinib, lorlatinib, entrectinib, crizotinib, ceritinib, brigatinib, osimeritinib, icotinib, gefitnib, erlotinib, erbitux, or any combination thereof.

In another aspect, anti-PD-1 antibodies or antigen-binding fragments thereof of the present disclosure may be used in a method of treating a viral infection, comprising administering to a patient in need thereof, an effective amount of an anti-PD-1 antibody or antigen binding fragment of the present disclosure, or a pharmaceutical composition comprising an anti-PD-1 antibody or antigen binding fragment of the present disclosure. In some embodiments, the viral infection is an acute or chronic viral infection. The ability of the anti-PD-1 antibodies or antigen-binding fragments thereof of the present disclosure to activate T-cells would be useful to treat chronic infections. Infectious viruses include eukaryotic viruses, such as adenovirus, bunyavirus, herpesvirus, papovavirus, papillomavirus (e.g., HPV), paramyxovirus, picornavirus, rhabdovirus (e.g., Rabies), orthomyxovirus (e.g., influenza), poxvirus (e.g., Vaccinia), reovirus, retrovirus, lentivirus (e.g., HIV), flavivirus (e.g., HCV, HBV), coronavirus (e.g., SARS-CoV-1, SARS-CoV-2), or the like.

In some embodiments, a viral infection to be treated with the anti-PD-1 antibodies or antigen-binding fragments thereof of the present disclosure is caused by HIV, hepatitis (A, B, or C), herpes virus (e.g., VZV, HSV-1, HAV-6, HSV-II, and CMV, Epstein Barr virus), adenovirus, influenza virus, flaviviruses, echovirus, rhinovirus, coxsackie virus, coronavirus, respiratory syncytial virus, mumps rubulavirus, rotavirus, measles morbillivirus, rubella virus, parvovirus, vaccinia virus, HTLV virus, dengue virus, papillomavirus, molluscum virus, poliovirus, rabies virus, JC virus, or arboviral encephalitis virus.

In some embodiments, an anti-PD-1 antibody or antigen binding fragment thereof described herein is used to treat a viral infection in combination with one or more anti-viral agents. In some embodiments, the one or more anti-viral agents are administered simultaneously, separately, or sequentially.

### EXAMPLES

### Example 1

### Identification and Characterization of Anti-PD1 Antibodies

### Materials and Methods

Antibody screening from a human ScFv library by phage display A human scFv phage display library was screened to identify phage antibodies that bound recombinant human PD-1-Fc fusion protein. Phages that bound to human Fc were depleted from the libraries by pre-incubation steps during the biopanning. Panned phages were selected based on binding to both human and cynomolgus PD-1. Their DNA sequences were cloned into a human expression vector with an IgG1 backbone containing the E233P, L234A, L235A, 4236, A327G, A330S and P331S mutations to nullify Fc-mediated effector functions (ADCC and CDC). These full IgG1 antibody clones were produced in Chinese hamster ovary (CHO) cells and purified using Protein A affinity column. Clones #53 and #61 were selected from a large number of functional hits based on binding, blocking, and in-vitro functional properties.

### PD-1 Binding ELISA (Enzyme-Linked Immunosorbent Assay)

A 96-well plate was coated with 25 ng (50 μl volume) of recombinant human PD-1-Fc, murine PD-1-Fc, or cynomolgus monkey PD-1-Fc fusion proteins (R&D Systems, Minneapolis, Minn.) overnight at 4° C. Wells were blocked for 1 h with 1% BSA and then washed once with PBS containing 0.1% Tween-20. Serially diluted anti-PD-1 antibodies or control IgG at 50 μl were then added and incubated at room temperature for 2 h. After washing, the plate was incubated with goat HRP-conjugated anti-human IgG Fab at RT for 1 h. The plates were washed and then incubated with 3, 3′,5,5′-tetramethylbenzidine. The absorbance at 450 nm was read on a microplate reader. EC50 (the half maximal effective concentration) was calculated.

### SPR Analysis of Antibody Binding Kinetics

The binding kinetics of the anti-PD-1 antibodies was measured using a Molecular Affinity Screening System (MASS-2, Sierra Sensors, Hamburg, Germany). Recombinant human, cynomolgus or mouse PD-1 protein was covalently immobilized onto separate flow cells on a High Capacity Amine sensorchip (Sierra Sensors) by amine coupling. The antibodies were injected at concentrations ranging from 100 nM to 0.004 nM. Sensorgrams were obtained at each concentration and the rate constants, the association rate (kₒₙ), and the dissociation rate (k_{off}) were evaluated. The affinity constant (K_{D}) was calculated from the ratio of k_{off}/kₒₙ.

### ELISA Assays on Blocking of PD-1 Interactions With PD-L1 or PD-L2

Serially diluted PD-1 antibodies or control IgG were mixed with a fixed amount of biotinylated PD-1-Fc (50 ng/ml), and then incubated at room temperature for 1 h. 100 μl of the mixture was transferred to 96-well plates pre-coated with recombinant human PD-L1-Fc or with human PD-L2-Fc fusion protein at 50 ng/well (R&D Systems) and then incubated at room temperature for an additional 1 h. Plates were washed; streptavidin-HRP conjugate was then added. The absorbance at 450 nm was measured. The concentration required for 50% inhibition (IC₅₀) of ligand-receptor interactions was calculated.

### Mixed Leukocyte Reaction (MLR) Assays to Measure the Ability of PD-1 Antibodies to Enhance T Cell Activation

CD4+ T cells were isolated from a healthy donor using EasySep Human CD4+ T cell isolation kit (Stemcell Technologies, Vancouver, BC). Peripheral blood mononuclear cells (PBMC) from a different healthy donor (Stemcell Technologies) were incubated in RPMI1640 medium with 10% fetal bovine serum at 37° C. at 5% CO₂ for 3 h. Non-adherent cells were removed and the remaining adherent cells were used for dendritic cell generation by culturing for 4 days in RPMI1640 medium with 10% fetal bovine serum containing IL-4 (20 ng/mL) and granulocyte-macrophage colony-stimulating factor (20 ng/mL, R&D systems).

For MLR assay, CD4+ T cells were mixed with the allogenic DC at a ratio of 10:1 in AIM-V medium containing 0.5% BSA. PD-1 antibodies or IgG control were then added at serially-diluted concentrations ranging from 100 μg/mL to 00.1 μg/mL. After 4 days of culture, supernatants were harvested and measured for IFN-γ production by an ELISA assay.

### PD-1 Luciferase Reporter Assay

A pair of engineered cell lines were used in this assay: 1) The PD-1⁺ Effector Cells (GloResponse NFAT-luc2/PD1 Jurkat cells), which are Jurkat T cells expressing PD-1 and a luciferase reporter driven by the NFAT response element; and 2) PD-L1⁺ Antigen Presenting Cells (PD-L1 aAPC/CHO-K1 Cells), which are CHO-K1 cells expressing PD-L1 and an engineered cell surface TCR-activating protein. When the two cell types are cultured together, the PD-1/PD-L1 interaction reduces TCR signaling and NFAT-mediated luminescence. Addition of antagonist PD-1 antibodies blocking the PD-1/PD-L1 interaction removes the co-inhibitory signal and leads to enhanced TCR activation and NFAT-RE-mediated luminescence.

PD-L1 aAPC/CHO-K1 human T-activator cells (Promega) were plated at 40,000 cells per well in a 96-well white opaque plate in 100 μl of RPMI-1640 medium with 10% FBS and incubated overnight at 37° C. at 5% CO₂. Medium was removed from the assay plate the following day and various concentrations of PD-1 antibodies and control antibody were added at 40 μl assay buffer per well. GloResponse NFAT-luc2/PD1 Jurkat cells (Promega) were re-suspended at 1.25×10⁶ /ml in assay buffer and added to the plate at 40 μl per well. After 6 h of incubation, assay plates were equilibrated at room temperature for 5 min. Bio-Glo™ Reagent (Promega) was added to each well at 80 μl per well. Plates were then incubated for 5 min at room temperature. Luminescence was measured in a plate reader.

### In Vivo Tumor Model

A mouse colon carcinoma MC38 model in human PD-1 knock-in C57/BL6 mice was used to evaluate the in vivo immune function enhancing activity of PD-1 antibodies. This mouse model contains a fully functioning mouse immune system. MC38 cell line expresses mouse PD-L1, and human PD-1 is known to be able to interact functionally with mouse PD-L1 ligand. Male mice of 6-8 weeks age were inoculated subcutaneously at the right flank with 5×10⁵ MC38 cells. When tumors reached an average volume of 100 mm³, the animals were randomly assigned into 4 groups (10 mice per group). Groups of mice were treated twice weekly with intraperitoneal administration of human IgG, Nivolumab (OPDIVO™, anti-PD-1 antibody) (Purchased from Bristol-Myers Squibb), Ab #53, or Ab #61 at 10 mg/kg. Tumor growth was monitored by measurement.

Results

### Anti-PD-1 Antibodies Ab #53 and Ab #61 Bind to PD-1 With High Affinity

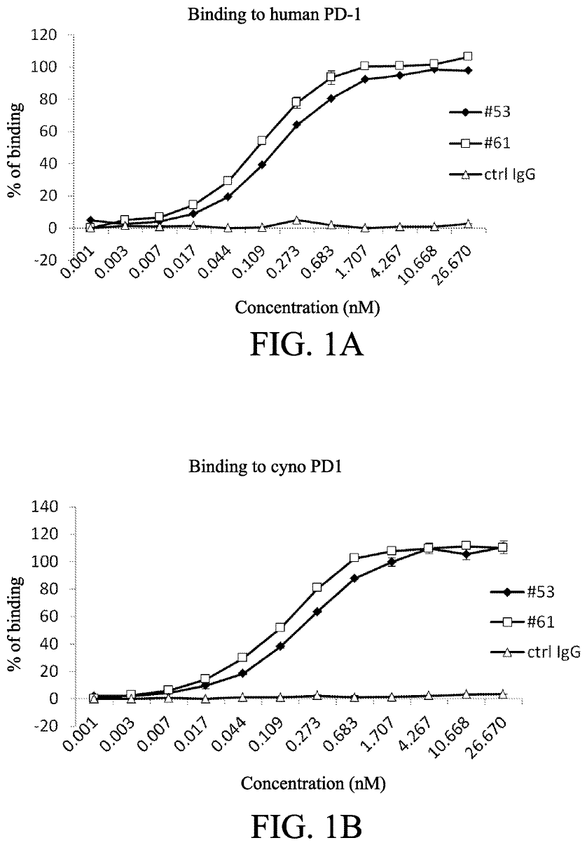

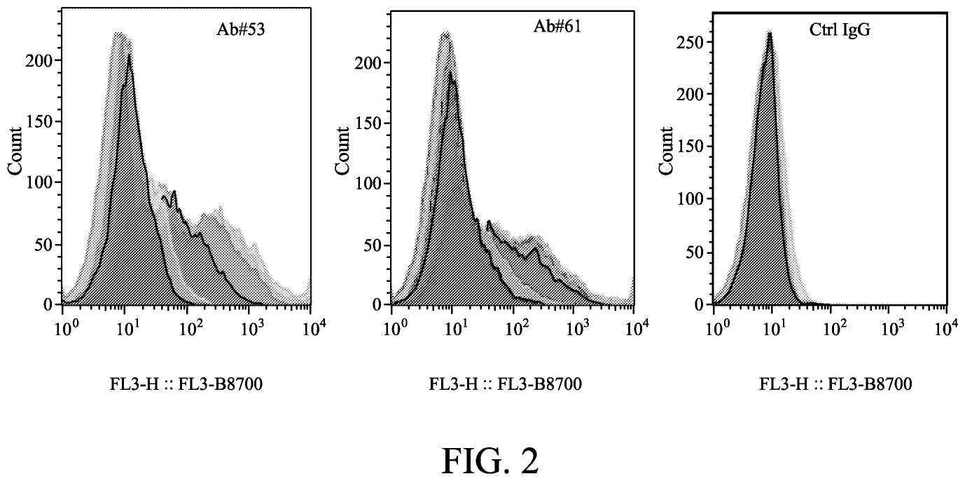

Several human anti-PD-1 antibody clones were identified by screening a human scFv library by phage display. These clones were constructed into a human IgG1 backbone containing mutations on Fc fragment that nullify FcRγ-mediated immune effector functions (ADCC and CDC). In ELISA assays, both Ab #53 and Ab #61 showed strong binding to recombinant human PD-1-Fc, with EC50 values of 0.19 nM and 0.08 nM, respectively (FIG. 1A). They also showed strong binding activities to recombinant cynomolgus monkey PD-1-Fc, with EC50 values of 0.24 nM and 0.11 nM, respectively (FIG. 1B). Ab #53 and Ab #61 bound strongly to cell surface PD-1, as a flow cytometric analysis showed binding to a human PD-1 transfected cell line (Jurkat-PD1) in a dose-dependent manner (FIG. 2).

The binding kinetics of anti-PD-1 antibodies to human, cynomolgus or mouse PD-1 were measured using SPR analysis. The affinities (K_{D}) of Ab #53 and Ab #61 to human PD-1 were determined to be 0.047 nM and 0.609 nM, with K_{off} values of 1.09×10⁻⁵ l/s and 1.72×10⁻⁴ l/s, respectively. Their affinities to cynomolgus PD-1 were found to be in the similar range, with a K_{D} of 0.29 nM and a K_{off} of 2.61×10⁻⁴ l/s for Ab #61 and a K_{D} of 3.33 nM and a K_{off} of 2.78×10⁻⁴ l/s for Ab #61 (Table 1). There was no binding to mouse PD-1 for either Ab #61 or Ab #53.

TABLE 1
Binding Kinetic Parameters of Anti-
PD-1 Antibodies by SPR analysis

| Antibody | Kₒₙ [1/(M · s)] | K_{off} [1/s] | K_{D} [M] |
|---|---|---|---|
| Ab#53: | | | |
| Human PD-1 binding | 2.44E+05 | 1.09E−05 | 4.47E−11 |
| Cynomolgus PD-1 | 8.99E+04 | 2.61E−05 | 2.90E−10 |
| binding | | | |
| Ab#61: | | | |
| Human PD-1 binding | 2.83E+05 | 1.72E−04 | 6.09E−10 |
| Cynomolgus PD-1 | 8.35E+04 | 2.78E−04 | 3.33E−09 |
| binding | | | |

### Blocking Activities on PD-1 Interactions to PD-L1 or PD-L2

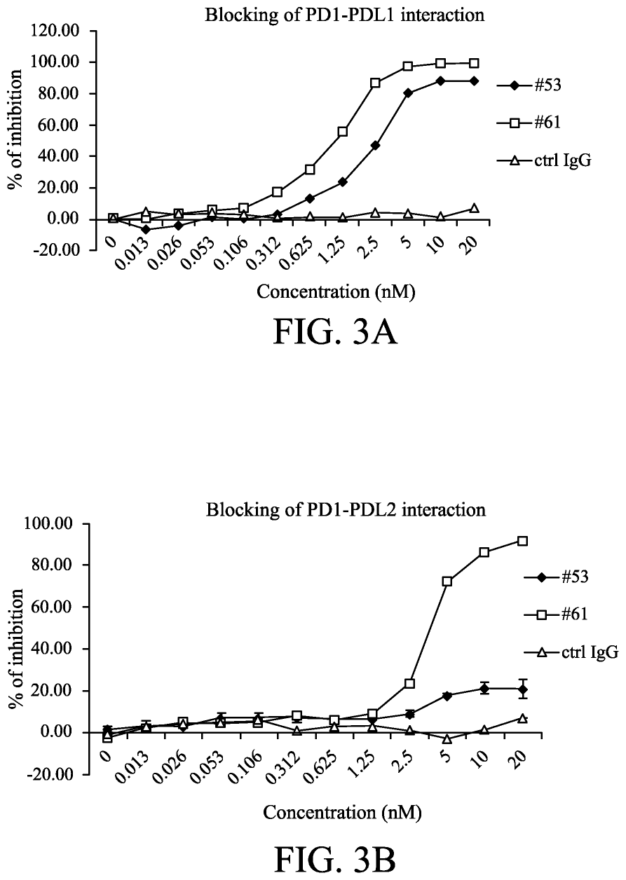

The ability of anti-PD-1 antibodies to block receptor-ligand interactions was examined in ELISA blocking assays. Ab #61 blocked the interactions of PD-1 to PD-L1 or PD-L2 with IC₅₀ values of 0.8 nM and 2.8 nM, respectively (FIGS. 3A-3B). Ab #53 showed strong blocking activity on the PD-1 interaction to PD-L1 (IC₅₀=2.4 nM) but no significant blocking on the PD-1 interaction to PD-L2 (FIGS. 3A-3B).

### Enhancement of T Cell Activation by Anti-PD-1 Antibodies In Vitro

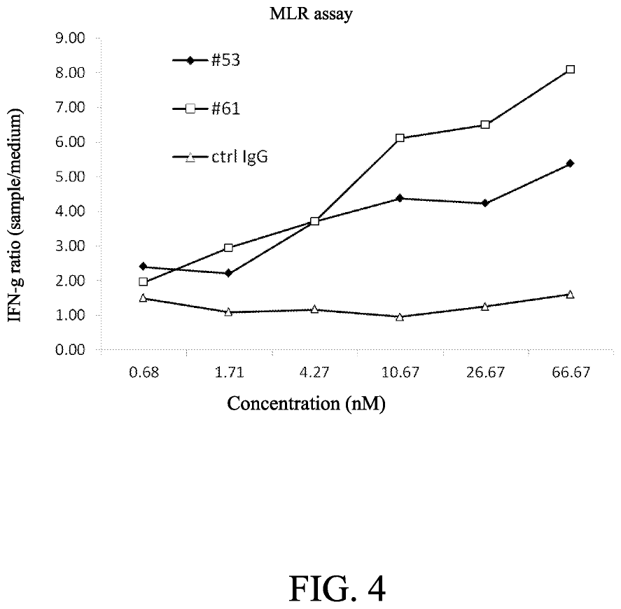

The ability of anti-PD-1 antibodies to enhance T cell activation was assessed using two different in vitro cell-based immune functional assays. In mixed leukocyte reactions using allogeneic human DC and T cells, both Ab #53 and Ab #61 induced a dose-dependent increase in allogenic T-cell response as measured by IFN-γ production by activated T cells (FIG. 4).

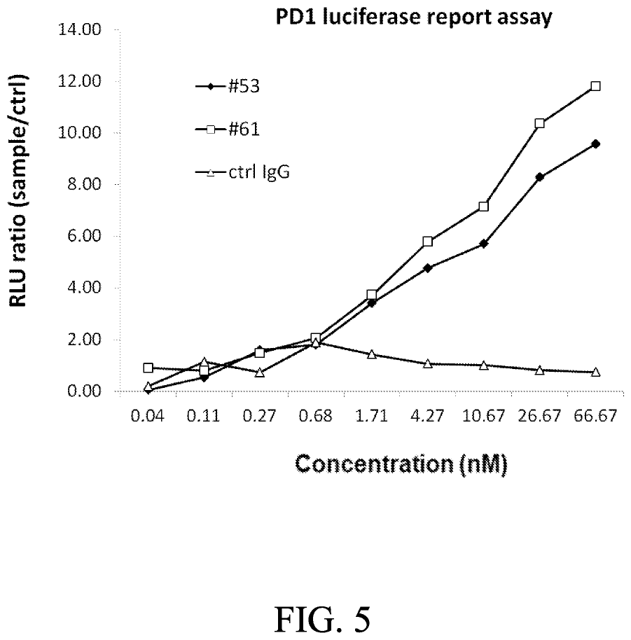

In the PD-1 luciferase reporter assay, addition of anti-PD-1 Ab #53 or Ab #61 resulted in enhanced T cell activation in a dose-dependent manner, with a maximal increase of 8.5 to 12 folds. These results indicated that both antibodies can block PD-1/PD-L1 interaction and enhance T cell function in the presence of TCR activation (FIG. 5).

### In Vivo Anti-Tumor Activities

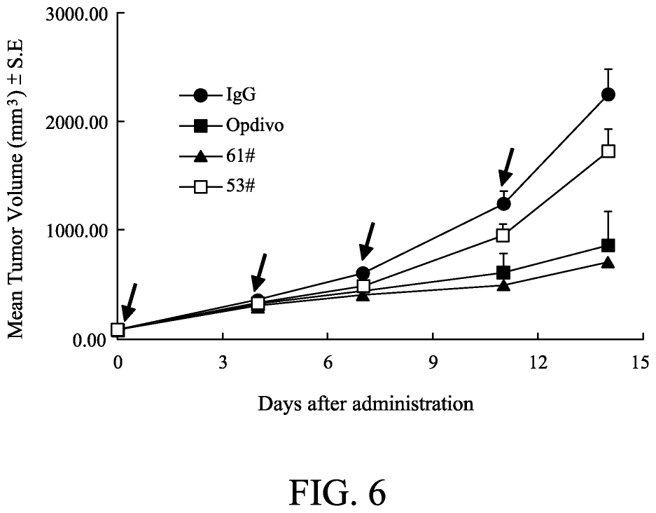

The in vivo anti-tumor efficacy of anti-PD-1 antibodies was evaluated using a syngeneic MC38 mouse tumor model in human PD-1 knock-in mice. Compared to untreated and human IgG-treated animals, treatment with Ab #61 resulted in significant tumor inhibition (p<0.01). Treatment with Ab #53 slightly delayed tumor growth compared to the IgG group (FIG. 6).

### Discussion

The discovery and characterization of two fully IgG1 antagonist anti-PD-1 antibodies are described.

Both Ab #53 and Ab #61 are fully human monoclonal antibody identified from a human scFv phage library. The human IgG1 Fc region of these antibodies contains the E233P, L234V, L235A, AG236, A327G, A330S, and P331S mutations to attenuate Fc-mediated effector functions (ADCC and CDC). Both antibodies bind to human and cynomolgus PD-1 with similar affinities as measured in both solid based ELISA and SPR analysis.

In two different cell-based immune functional assays, it is found that both anti-PD-1 antibodies were able to enhance T cell activation. In the allogeneic MLR assay, both Ab #53 and Ab #61 strongly enhanced T cell activation. In the PD-1 luciferase reporter assay performed in the presence of TCR activation and PD-1/PD-L1 engagement, both antibodies were able to effectively block the PD-1/PD-L1 interaction and hence enhanced TCR-mediated signaling in the effector T cells.

The syngeneic MC38 mouse colon carcinoma model in human PD-1 knock-in mice was used to evaluate the in vivo anti-tumor efficacy of our anti-PD-1 antibodies. Treatment with weekly doses of Ab #61 at 10 mg/kg strongly inhibited tumor growth in these mice.

In conclusion, the data demonstrated Ab #61 is a potent anti-PD-1 antibody with favorable in vitro and in vivo properties. Ab #53 is a unique anti-PD-1 antibody which blocks PD-1/PD-L1 but not PD-1/PD-L2 interaction.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Patent Application No. 63/000,386, filed on Mar. 26, 2020, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An isolated antibody or an antigen-binding fragment thereof, that specifically binds to PD-1, wherein the antibody or antigen-binding fragment thereof comprises a heavy chain variable region (VH) and a light chain variable region (VL), wherein
(a) the VH comprises a heavy chain CDR1 (VH-CDR1) comprising the amino acid sequence of SEQ ID NO:1, a heavy chain CDR2 (VH-CDR2) comprising the amino acid sequence of SEQ ID NO:2, and a heavy chain CDR3 (VH-CDR3) comprising the amino acid sequence of SEQ ID NO:3; and the VL comprises a light chain CDR1 (VL-CDR1) comprising the amino acid sequence of SEQ ID NO:4, a light chain CDR2 (VL-CDR2) comprising the amino acid sequence of SEQ ID NO:5, and a light chain CDR3 (VL-CDR3) comprising the amino acid sequence of SEQ ID NO:6; or
(b) the VH comprises a VH-CDR1 comprising the amino acid sequence of SEQ ID NO:7, a VH-CDR2 comprising the amino acid sequence of SEQ ID NO:8, and a VH-CDR3 comprising the amino acid sequence of SEQ ID NO:9, and the VL comprises a VL-CDR1 comprising the amino acid sequence of SEQ ID NO:10, a VL-CDR2 comprising the amino acid sequence of SEQ ID NO:11, and a VL-CDR3 comprising the amino acid sequence of SEQ ID NO:12.

2. The antibody or antigen binding fragment thereof of claim 1, wherein
(a) the VH comprises an amino acid sequence that has at least 90% identity with the amino acid sequence of SEQ ID NO:13, and the VL comprises an amino acid sequence that has at least 90% identity with the amino acid sequence of SEQ ID NO:15; or
(b) the VH comprises an amino acid sequence that has at least 90% identity with the amino acid sequence of SEQ ID NO:17, and the VL comprises an amino acid sequence that has at least 90% identity with the amino acid sequence of SEQ ID NO:19.

3. The antibody or antigen binding fragment thereof of claim 2, wherein
(a) the VH comprises the amino acid sequence of SEQ ID NO:13, and the VL comprises the amino acid sequence of SEQ ID NO:15; or
(b) the VH comprises the amino acid sequence of SEQ ID NO:17, and the VL comprises the amino acid sequence of SEQ ID NO:19.

4. The antibody or antigen binding fragment thereof of claim 1, wherein the antibody comprises a Fc region or a variant thereof.

5. The antibody or antigen binding fragment thereof of claim 4, wherein the antibody comprises an IgG1 Fc region or variant thereof, optionally wherein the IgG1 Fc region comprises the amino acid sequence of SEQ ID NO:35 or SEQ ID NO:36.

6. The antibody or antigen binding fragment thereof of claim 1, wherein the antibody comprises a human IgG1, human IgG2, human IgG3, or human IgG4 constant region or a variant thereof.

7. The antibody or antigen binding fragment thereof of claim 1, wherein the antibody comprises a heavy chain (HC) and a light chain (LC), wherein:
(a) the HC comprises an amino acid sequence that has at least 90% identity with the amino acid sequence of SEQ ID NO:25, and the LC comprises an amino acid sequence that has at least 90% identity with the amino acid sequence of SEQ ID NO:26; or
(b) the HC comprises an amino acid sequence that has at least 90% identity with the amino acid sequence of SEQ ID NO:27, and the LC comprises an amino acid sequence that has at least 90% identity with the amino acid sequence of SEQ ID NO:28.

8. The antibody or antigen binding fragment thereof of claim 7, wherein:
(a) the HC comprises the amino acid sequence of SEQ ID NO:25, and the LC comprises the amino acid sequence of SEQ ID NO:26; or
(b) the HC comprises the amino acid sequence of SEQ ID NO:27, and the LC comprises the amino acid sequence of SEQ ID NO:28.

9. The antibody or antigen binding fragment thereof of claim 1 wherein:
(a) the antibody is a humanized antibody; or
(b) the antibody is glycosylated.

10. (canceled)

11. The antibody or antigen binding fragment thereof of claim 1 wherein the antibody has:
(a) a normal effector function; or
(b) a reduced effector function.

12. (canceled)

13. A pharmaceutical composition comprising an antibody or antigen-binding fragment thereof of claim 1 and a pharmaceutically acceptable carrier.

14. An isolated nucleic acid that encodes the HC and/or LC of the antibody or antigen-binding fragment thereof claim 1.

15. A vector comprising the nucleic acid of claim 14.

16. An isolated host cell comprising the vector of claim 15.

17. An isolated host cell that expresses the antibody or antigen-binding fragment thereof claim 1.

18. A mammalian host cell comprising:
(a) a polynucleotide sequence encoding a polypeptide having the amino acid sequence of SEQ ID NO:13 and a polynucleotide sequence encoding a polypeptide having an amino acid sequence of SEQ ID NO:15;
(b) a polynucleotide sequence encoding a polypeptide having the amino acid sequence of SEQ ID NO:17 and a polynucleotide sequence encoding a polypeptide having an amino acid sequence of SEQ ID NO:19;
(c) a polynucleotide sequence encoding a polypeptide having the amino acid sequence of SEQ ID NO:25 and a polynucleotide sequence encoding a polypeptide having an amino acid sequence of SEQ ID NO:26; or
(d) a polynucleotide sequence encoding a polypeptide having the amino acid sequence of SEQ ID NO:27 and a polynucleotide sequence encoding a polypeptide having an amino acid sequence of SEQ ID NO:28,
wherein the cell is capable of expressing an antibody or antigen-binding fragment thereof that binds human PD-1.

19. A method of producing an antibody or antigen-binding fragment thereof that binds PD-1, comprising culturing the host cell of claim 16 under conditions suitable for expressing the antibody or antigen-binding fragment thereof.

20. (canceled)

21. A method of treating cancer or a viral infection comprising administering to a patient in need thereof an effective amount of the antibody or antigen-binding fragment thereof of claim 1.

22. The method of claim 21, wherein the cancer is a hematologic malignancy, a solid tumor, Hodgkin's lymphoma, non-Hodgkin's lymphoma, multiple myeloma, leukemia, myelodysplastic syndrome, thymus cancer, malignant mesothelioma, pituitary tumor, thyroid tumor, melanoma, Merkel cell skin cancer, lung cancer, head and neck cancer, colorectal cancer, liver cancer, bile duct cancer, gallbladder cancer, pancreatic cancer, esophageal cancer, gastric cancer, small intestine cancer, anal cancer, kidney cancer, bladder cancer, prostate cancer, penile cancer, testicular cancer, breast cancer, ovarian cancer, cervical cancer, vaginal cancer, vulvar cancer, endometrial cancer, eye cancer, soft tissue sarcoma, or hepatocellular carcinoma, brain tumor, or spinal cord tumor.

23. (canceled)

24. The method of claim 21, further comprising administering one or more anti-tumor agents simultaneously, separately, or sequentially.

25. (canceled)


SEQUENCE LISTING (36 sequences)

SEQ ID NO: 1 - protein, 10 aa, Homo sapiens
  Ab#53 VH-CDR1
GFTFSSYAMS

SEQ ID NO: 2 - protein, 9 aa, Homo sapiens
  Ab#53 VH-CDR2
ISGSGGSTY

SEQ ID NO: 3 - protein, 6 aa, Homo sapiens
  Ab#53 VH-CDR3
WGAFDI

SEQ ID NO: 4 - protein, 6 aa, Homo sapiens
  Ab#53 VL-CDR1
QSIRIH

SEQ ID NO: 5 - protein, 3 aa, Homo sapiens
  Ab#53 VL-CDR2
DTS

SEQ ID NO: 6 - protein, 9 aa, Homo sapiens
  Ab#53 VL-CDR3
QQYYAWPLT

SEQ ID NO: 7 - protein, 10 aa, Homo sapiens
  Ab# 61 VH-CDR1
GFTFSSYAMS

SEQ ID NO: 8 - protein, 9 aa, Homo sapiens
  Ab# 61 VH-CDR2
ISGSGGSTY

SEQ ID NO: 9 - protein, 10 aa, Homo sapiens
  Ab# 61 VH-CDR3
ASPDYGELRY

SEQ ID NO: 10 - protein, 6 aa, Homo sapiens
  Ab# 61  VL-CDR1
LAISGW

SEQ ID NO: 11 - protein, 3 aa, Homo sapiens
  Ab# 61  VL-CDR2
GAT

SEQ ID NO: 12 - protein, 9 aa, Homo sapiens
  Ab# 61  VL-CDR3
QQYYDYPLT

SEQ ID NO: 13 - protein, 113 aa, Homo sapiens
  Ab#53 VH
EVQLLESGGGLVQPGGSLRLSCAASGFTFSSYAMSWVRQAPGKGLEWVSAISGSGGSTYYADSVKGRFTISRDNYKNTLYLQMNSMRAEDTAVYYCWGAFDIWGQGTMVTVSS

SEQ ID NO: 14 - DNA, 339 bp, Homo sapiens
  Ab#53 VH
gaggtgcagctgttggagtctgggggaggcttggtacagcctggggggtccctgagactctcctgtgcagcctctggattcacctttagcagctatgccatgagctgggtccgccaggctccagggaaggggttggagtgggtctcagctattagtggtagtggtggtagcacatactacgccgactccgtgaagggccggttcaccatctccagagacaattacaagaacacgctgtatttgcaaatgaacagcatgagagccgaggacacggccgtatattactgttggggggcttttgatatctggggccaaggaaccatggtcaccgtctcctca

SEQ ID NO: 15 - protein, 107 aa, Homo sapiens
  Ab#53 VL
DIQLTQSPATLSASAGEGATLSCRASQSIRIHVAWYQQKPGQAPRLLIYDTSTRATGIPARFSGSGSGTEFTLTISSLQLEDFAVYYCQQYYAWPLTFGGGTKVEIK

SEQ ID NO: 16 - DNA, 321 bp, Homo sapiens
  Ab#53 VL
gacatccagttgacccagtctccagccactctgtctgcgtctgcaggggaaggagccaccctctcctgtagggccagtcagagtattagaatccacgtagcctggtaccagcagaaacctggccaggctcccaggctcctcatctatgatacatccaccagggccacgggtatcccagccaggttcagtggcagtgggtctgggacagagttcactctcaccatcagcagcctgcagcttgaagattttgcagtttattactgtcagcagtattatgcctggcctctcactttcggcggcgggaccaaggtggagatcaaa

SEQ ID NO: 17 - protein, 117 aa, Homo sapiens
  Ab# 61 VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCASPDYGELRYWGQGTLVTVSS

SEQ ID NO: 18 - DNA, 350 bp, Homo sapiens
  Ab# 61 VH
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgagtcccgactacggtgaattgcgctactggggccagggaaccctggtcaccgtctcctc

SEQ ID NO: 19 - protein, 107 aa, Homo sapiens
  Ab# 61 VL
EIVMTQSPSSVSASVGDRVTITCRASLAISGWLAWYQQTPGKAPRLLIYGATTLQSGVPSRFSGSGSGTDFTLTISRLQSEDFATYYCQQYYDYPLTFGGGTKVEIK

SEQ ID NO: 20 - DNA, 321 bp, Homo sapiens
  Ab# 61 VL
gaaattgtaatgacacagtctccatcttctgtgtctgcatctgtaggagacagagtcaccatcacttgtcgggcgagtctggctattagcggctggttagcctggtatcagcagacaccagggaaagcccctaggctcctgatctatggtgcaaccacgttgcagagtggggttccatcacggttcagcggcagtggatctgggacagatttcactctcaccatcagccgcctgcagtctgaagattttgcaacttattactgtcaacaatattatgattaccctctcactttcggcggaggaaccaaggtggagatcaaa

SEQ ID NO: 21 - protein, 329 aa, Artificial Sequence
  IgG1 heavy chain constant region E233P, L234V, L235A, delta236, A327G, A330S, and P331S variant
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - DNA, 987 bp, Artificial Sequence
  IgG1 heavy chain constant region E233P, L234V, L235A, delta236, A327G, A330S, and P331S variant
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccttgcccagcaccacctgtggcaggaccttcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 23 - protein, 107 aa, Homo sapiens [sequence omitted]

SEQ ID NO: 24 - DNA, 321 bp, Homo sapiens
  LC kappa constant domain
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaagacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 25 - protein, 442 aa, Artificial Sequence
  Ab#53 heavy chain
EVQLLESGGGLVQPGGSLRLSCAASGFTFSSYAMSWVRQAPGKGLEWVSAISGSGGSTYYADSVKGRFTISRDNYKNTLYLQMNSMRAEDTAVYYCWGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 214 aa, Homo sapiens
  Ab#53 light chain
DIQLTQSPATLSASAGEGATLSCRASQSIRIHVAWYQQKPGQAPRLLIYDTSTRATGIPARFSGSGSGTEFTLTISSLQLEDFAVYYCQQYYAWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 446 aa, Artificial Sequence
  Ab# 61 heavy chain
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCASPDYGELRYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 214 aa, Homo sapiens
  Ab# 61 light chain
EIVMTQSPSSVSASVGDRVTITCRASLAISGWLAWYQQTPGKAPRLLIYGATTLQSGVPSRFSGSGSGTDFTLTISRLQSEDFATYYCQQYYDYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 330 aa, Homo sapiens
  wildtype IgG1 heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - DNA, 990 bp, Homo sapiens
  wildtype IgG1 heavy chain constant region
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccttgcccagcaccagaactcctggggggaccttcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 31 - protein, 113 aa, Homo sapiens
  IgG1 CH1 + hinge
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCP

SEQ ID NO: 32 - DNA, 339 bp, Homo sapiens
  IgG1 CH1 + hinge
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccttgccca

SEQ ID NO: 33 - protein, 327 aa, Homo sapiens
  IgG4 constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 15 aa, Homo sapiens
  IgG1 hinge
EPKSCDKTHTCPPCP

SEQ ID NO: 35 - protein, 217 aa, Homo sapiens
  wildtype IgG1 Fc region
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 216 aa, Artificial Sequence
  IgG1 Fc region E233P, L234V, L235A, delta236, A327G, A330S, and P331S variant
APPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK